(19) United States
(12) Patent Application Publication
(10) Pub. No.: US 2006/0026712 A1
(43) Pub. Date: Feb. 2, 2006

(54) Method for the generation of green-note compounds

(76) Inventors: David Hildebrand, Lexington, KY (US); Hirotada Fukushige, Lexington, KY (US)

Correspondence Address: MCDERMOTT WILL & EMERY LLP, 600 13TH STREET, N.W., WASHINGTON, DC, 20005-3096, US

(21) Appl. No.: 10/900,408

(22) Filed: Jul. 28, 2004

(51) Int. Cl. C12P 7/24; C12N 15/82; A01H 1/00; A01H 5/00

(52) U.S. Cl. 800/281; 800/317.3; 435/147

(57) ABSTRACT

Methods and systems for generating green-note compounds, particularly leaf aldehyde, (2E)-hexenal are provided. The methods include the use of plants with enhanced hydroperoxide lyase (HL) activity and soybean seed meal or flour from soybean lipoxygenase mutants to enhance (2E)-hexenal production.

### FIELD OF THE INVENTION

This invention relates to methods and systems for generating green-note compounds, particularly leaf aldehyde, (2E)-hexenal. More particularly, the invention relates to the use of tobacco plants with enhanced hydroperoxide lyase (HL) activity and soybean seed meal from soybean lipoxygenase mutants to enhance (2E)-hexenal production.

### BACKGROUND OF THE INVENTION

The six-carbon (C₆) volatile compounds, such as hexenal, (2E)-hexenal, and (3Z)-hexenol, are the most abundant lipid peroxidation products formed in plant tissues. A key enzyme in fatty acid (FA) peroxidation is lipoxygenase [linoleate: oxygen oxidoreductase] (LOX). LOXs are a class of dioxygenases that catalyze the peroxidation of polyunsaturated FAs and other molecules containing cis, cis-114-pentadiene moieties. These polyunsaturated FAs such as linolenic (18:3) or linoleic acid (18:2) can be converted by LOXs into hydroperoxy FAs with the hydroperoxy group positioned either on the 9th (9-) or 13th (13-) carbon of the 18 carbon chain. LOXs can also peroxidize FAs esterified in glycerolipids such as phospholipids and apparently glycolipids, but free FAs are the preferred substrate for most LOXs at least in vitro. The FA hydroperoxides resulting from LOX action can be metabolized by one of several major pathways operative in higher plant tissues. In one pathway, HL catalyzes the cleavage of 13-hydroperoxy 18:2 (13-HPOD) or 18:3 (13-HPOT) into the 12 carbon compound, 12-oxo-(9Z)-dodecenoic acid, and the C₆ aldehyde, hexanal or (3Z)-hexenal. Nine carbon oxo-FAs and aldehydes can be formed from the 9-hydroperoxy FAs. The 12-oxo-(9Z)-dodecenoic acid and (3Z)-hexenal undergo isomerization to the more stable 12-oxo-(10E)-dodecenoic acid and (2E)-hexenal. Small levels of (3E)-hexenal are sometimes formed. Hexenals are frequently converted to their corresponding alcohols by alcohol dehydrogenase (ADH). The 12-oxo-(10E)-dodecenoic acid, known as traumatin or wound hormone, is readily oxidized non-enzymatically to (2E)- dodecenedioic acid, commonly known as traumatic acid, but this may be formed in vitro during extraction. The first HL cDNA to be cloned and sequenced is that for the bell pepper fruit HL (Matsui et al., 1996 FEBS Letters 394: 21-24). Many additional HLs have been identified and cloned from several plant species, such as Arabidopsis, tomatoes and potatoes, as well as tobacco and watermelon (Hildebrand et al., unpublished data).

The leaf aldehyde and alcohols [(3Z)-hexenol] are important components of the aroma and flavor of fruits and vegetables, such as apples, bananas and tomatoes (Drawert et al., 1966 Ann Chem 694: 200-208; Kazeniac and Hall, 1970 Journal of Food Science 35: 519-530), and are associated with ‘green notes’ of leaves (Hatanaka et al., 1987 Chemistry and Physics of Lipids 44: 341-361; Gaidner, 1989 In D B Min, T H Smouse, eds, Flavor Chemistry of Lipid Foods. American Oil Chemists' Society, Champaign, Ill., pp 98-112). These volatile compounds can be lost during the production process (such as canning or drying) and are used as food and beverage flavorings to impart a green character and freshness. For some commodities such as soybeans, on the other hand, these C₆ aldehydes and alcohols produced during processing cause off-odors and/or off-flavors. Harb et al. (Harb et al., 1994 Acta Horticulturae 368: 142-149) showed the enhanced production of aromas from apples by supplying aroma precursors, such as hexanol, before or after long controlled air storage. Song et al. (Song et al., 1996 Acta Horticulturae 368: 142-149) showed the potential of hexanal as a residueless fungicide and flavor enhancer for post-harvest storage of apples. Archbold et al. (Archbold et al., 1999 Hort Science 34: 705-707) showed similar reductions of fungal infection of grapes by (2E)-hexenal.

Though the majority of these compounds have been chemically synthesized at industrial levels, there are increasing demands for natural flavors as well as natural chemicals for fungicides and pesticides as more and more consumers are interested in natural products, organic production and ‘green’ industries (Whitehead et al., 1995 Cereal Foods World 40:193-194). Commercial production of natural leaf aldehyde is achieved from watermelon leaves because of the high yield of this compound from this source. This involves making large-scale homogenates of watermelon leaves with added linolenic acid or hydrolyzed linseed. Whitehead et al. (Whitehead et al., 1995 Cereal Foods World 40:193-194) proposed the usage of soybean LOX to produce 13-HPOT in combination with plant materials to generate C₆ aldehydes as well as baker's yeast ADH to convert them to C₆ alcohols when desired.

Thus, there is a need for improved methods and systems for the production of natural green-note compounds.

### SUMMARY OF THE INVENTION

In one aspect of the invention there is provided a method for enhancing the production of green note compounds comprising homogenizing green leaves of hydroperoxide producing plants in the presence of fatty acid and soy linoleate oxygen oxidoreductase (LOX), wherein the soy lipoxygenase is LOX1, LOX2 or a combination of LOX1 and LOX2. In a preferred embodiment, the source of LOX is a soy bean seed flour or soy bean meal. In another preferred embodiment, the soy bean flour or soy bean meal prepared from LOX3 lacking seeds.

In another aspect of the invention there is provided a soy bean seed flour or soy bean meal prepared from soy bean seeds that lack LOX3.

### BRIEF DESCRIPTION OF THE DRAWINGS

FIG. 1 is an illustration of the oxylipin pathway in higher plants (18:3 as an initial substrate).

FIG. 2 is a bar graph showing the relative HL activity in the leaves of various plants.

FIG. 3 is a bar graph showing the HL activity in leaf tissue of transgenic and control tobacco compared to watermelon.

FIG. 4 is a bar graph showing the HL activity of line N24, a transgenic tobacco line, compared to watermelon.

FIG. 5 is a bar graph showing the effects of soy meal on the production of (3Z)-hexenal, (2E)-hexenal and (3Z)-hexenol in green pepper fruits.

FIG. 6 is a bar graph showing the effect of linolenic acid (18:3) and defatted flour of LOX mutant soybeans on C₆ aldehyde production in watermelon-HL over-expressing tobacco leaves.

FIG. 7 is a bar graph showing amount of C₆ aldehyde production from watermelon-HL over-expressing tobacco leaves that have been homogenized with fatty acid (18:3) and defatted flour of LOX mutant soybeans.

FIG. 8 is a bar graph showing the effects of pH on C₆ aldehyde production from watermelon-HL over-expressing tobacco leaves homogenized with fatty acid (18:3) and defatted flour of LOX mutant soybeans.

FIG. 9 is a bar graph showing the effects of pH and isozyme line on C₆ aldehyde production from watermelon-HL over-expressing tobacco leaves homogenized with 18:3 and defatted soy flour.

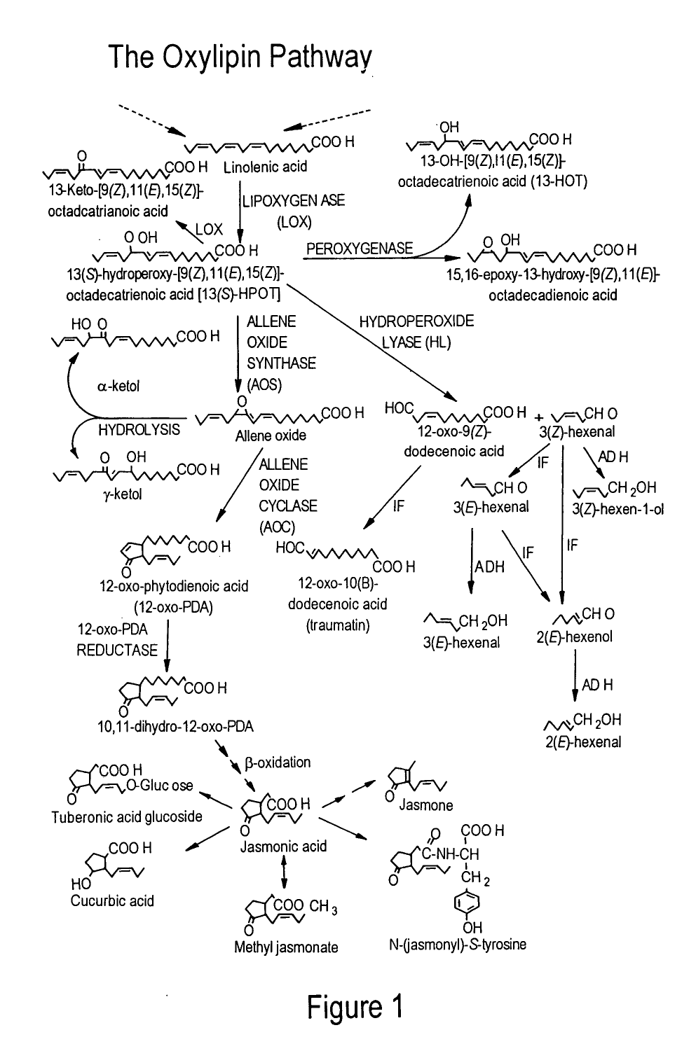

### DETAILED DESCRIPTION OF THE INVENTION

Methods and systems for enhancing the production of green note compounds, in particular leaf aldehyde, (2E)-hexenal, are described. It has been found that C₆ aldehyde production can be optimized by homogenizing hydroperoxide producing plants or plant tissue in the presence of a soybean powder, such as a flour or acetone powder prepared from ground seeds of soybean mutant isozyme lines or plants that produce only one isozyme of LOX, either LOX1 or LOX2, or which produce a combination of LOX1 and LOX2, but not LOX3. The present invention provides increased formation of leaf aldehydes via plant leaves by enhanced expression of the key enzyme involved in its formation and the usage of plant leaves to produce green note compounds.

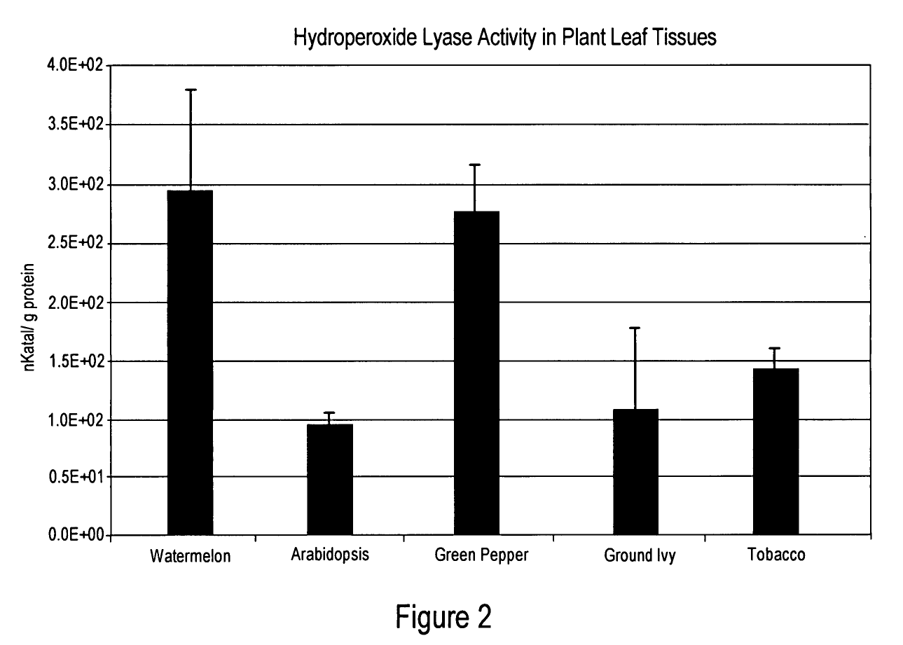

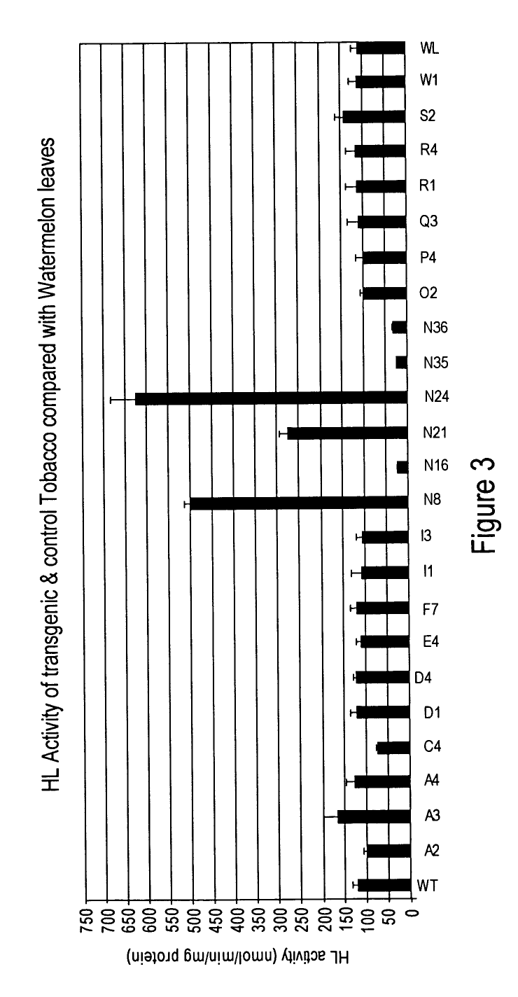

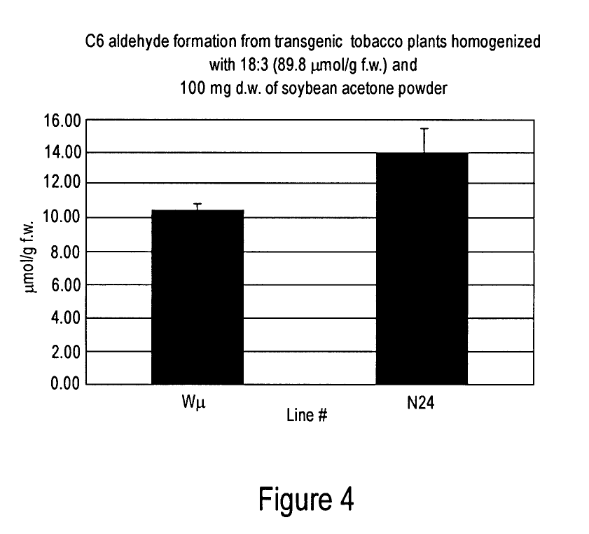

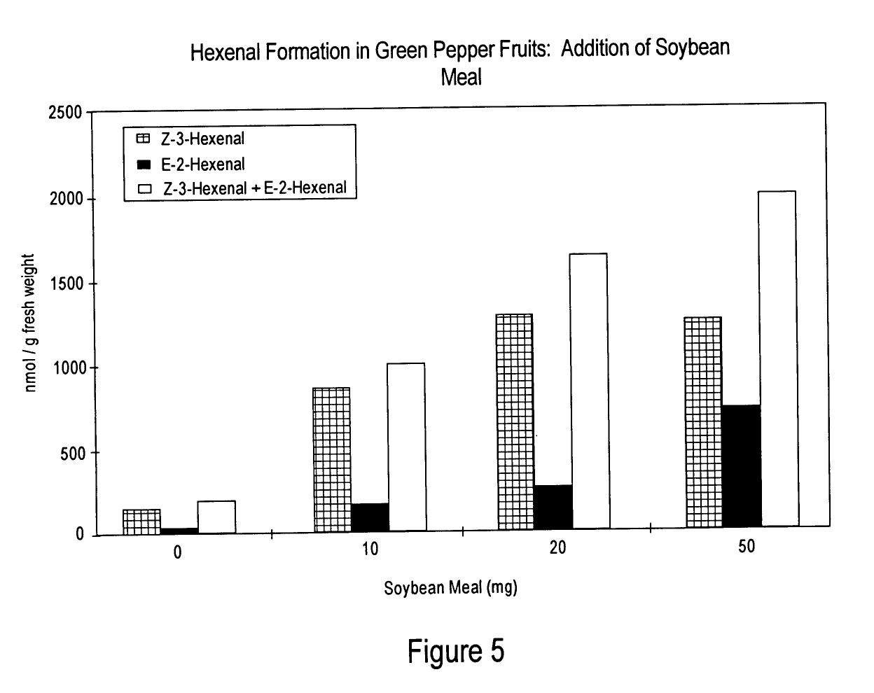

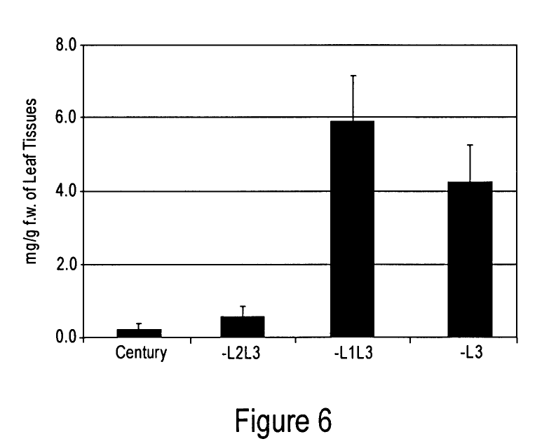

In order to better understand how to increase overall oxylipin formation with maximum flow to leaf aldehyde, natural variations in such processes were surveyed. Consistent with reports in the literature, we find watemelon (Citrullus lanatus) to be a high C₆-aldehyde producer. Green pepper (Capsicum annuum) leaf is also a high C₆-aldehyde producer. Arabidopsis had the lowest activity among five species examined. [FIG. 2].

Grinding the leaf tissues of C. lanatus and tobacco with 18:3 in water followed by a short incubation, e.g., one to thirty minute incubation, or by freezing them, results in high C₆-aldehyde levels [mostly (3Z)-hexenal]; and further incubation at high temperature accelerates isomerization to leaf aldehyde or (2E)-hexenal. As LOX appears to be a limiting factor for the C₆-aldehyde and alcohol formation in green leaves, effects of additional LOX were examined. LOX 3-null line soybean seeds were used as the source of exogenous LOX since LOX 3 was shown to metabolize 13-HPOD competing with HL for C₆-aldehyde and alcohol production (Hildebrand et al., 1990; Hildebrand et al., 1991). Also, LOX 3 largely produces HPOD or HPOT stereoisomers, which are not precursors for C₆-aldehyde formation. Grinding green tissues (watermelon leaf or green pepper fruit) with LOX3-null soybean meal or 18:3 increased C₆-aldehyde and alcohol formation. However, when soybean meal and green tissues were combined and ground with 18:3, the increase in C₆-aldehyde and alcohol formation was much greater than the mere sum of those of green tissues or soybean meal alone.

It is shown herein that the production of green note compounds from green leaves is enhanced by the addition of a single LOX isozyme, either LOX1 or LOX2, or the combination of LOX1 and LOX2, during homogenization of the leaves. In a preferred method of the invention, green leaves, such as those obtained from watermelon plants, or other green note producing plant, e.g., green peppers, tobacco, genetically engineered plants that overproduce HL, are ground in the presence of a fatty acid source, such as a hydrolyzed oil rich in linolenic acid or linoleic acid, e.g., linseed oil, sesame oil, soybean oil, and combinations thereof, and soy flour or soy bean meal. Preferably defatted, prepared flour or meal from soy bean plants or cell lines that produce only LOX1, LOX2 or a combination of LOX1 and LOX2 are used. In a preferred embodiment the LOX 1 and/or LOX2 isozyme is obtained from a soy plant or cell line that produces only one isozyme of LOX, or which does not produce LOX3 (LOX3 null). In a most preferred embodiment, the soy plant or cell line produces only LOX2.

C₆ aldehyde production from fatty acid, such as linolenic acid (18:3) or linoleic acid (18:2), is optimized by homogenizing leaf tissues in the presence of the fatty acid, soymeal or soy flour obtained from soy bean plants or cell lines that produce a single LOX isozyme (LOX1 or LOX2) or from plants or cells that produce only LOX1 and LOX2. When LOX2 alone is used, the preferred pH under which homogenization is carried out is about 6. When LOX1 alone is used the preferred pH is 9. The pH is adjusted through use of an appropriate buffer, for example, such as a phosphate buffer (pH6) or borate buffer (pH 9).

The present invention also provides soy meal and soy flour prepared from soy plants of cell lines that produce only a single LOX isozyme, either LOX1 or LOX2, or from plants or cell lines that produce a combination of LOX1 and LOX2, but not LOX3. Soybeans are ground in a grinder or mill to fine four. Soy flour, then, are defatted by washing with an ample volume of cold acetone (−20 C.) followed by an ample volume of cold diethyl ether (−20 C.). Defatted flour is dried under reduced oxygen atmosphere by, but not limited to a gentle stream of nitrogen gas.

Although illustrative embodiments of the present invention have been described in detail, it is to be understood that the present invention is not limited to those precise embodiments, and that various changes and modifications can be effected therein by one skilled in the art without departing from the scope and spirit of the invention as defined by the appended claims.

### EXAMPLES

### Example 1

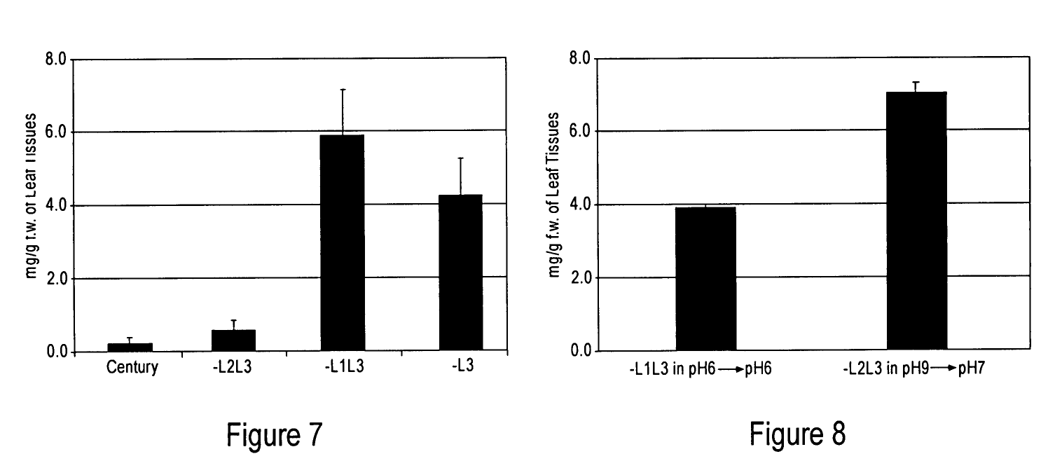

The effect of LOX isozyme lines was analyzed to optimize C₆ aldehyde production from 18:3, HL-overexpressing tobacco leaves and defatted soybean flour. 5.0 mg 18:3 were homogenized with 25 mg soy flour and 50 mg f.w. leaf tissues of Watermelon-HL overexpressing Nicotiana plants in 2.0 mL of water for 2.0 minutes. As seen in FIG. 7, the use of -L1 L3, which only contains LOX2, resulted in the highest production of C₆ aldehydes while Century, a wild type, reduced the production to 1/50^{th} of the level. LOX1 (-L2L3) produced less HPOT, the substrate of HL, compared to LOX2, potentially due to the acidity of leaf homogenates. LOX1 inclusion in the flours somewhat reduced the overall production in -L3.

### Example 2

Since LOX1 and LOX2 have different pH optimums (pH 9 and 6, respectively), buffered solutions were tested for optimum C₆ aldehyde production. For LOX1 (-L2L3), 2.0 mg of 18:3 and 5.0 mg of soy flour were homogenized in 2.0 mL of 50 mM pH9 borate buffer for 1.0 minute. Then the pH was lowered to about pH 7 by addition of 0.5 mL of 0.2 M KH₂PO₄ solution and further homogenized with 50 mg f.w. leaf tissues for 2.0 minutes. For LOX2 (-L1L3), soy flour was homogenized with 18:3 in 2.0 mL of pH 6.0 buffer and then 0.5 mL of pH6 buffer was added. The leaf tissues were added and homogenized. As seen in the FIG. 8, C₆ aldehyde production increased when LOX1 was homogenized in pH9 buffer as expected. On the other hand, when LOX2 was homogenized with 18:3 alone first, the production of C₆ aldehydes was lower than the level seen when 18:3, soy flour, and leaf tissues were homogenized together.

### Example 3

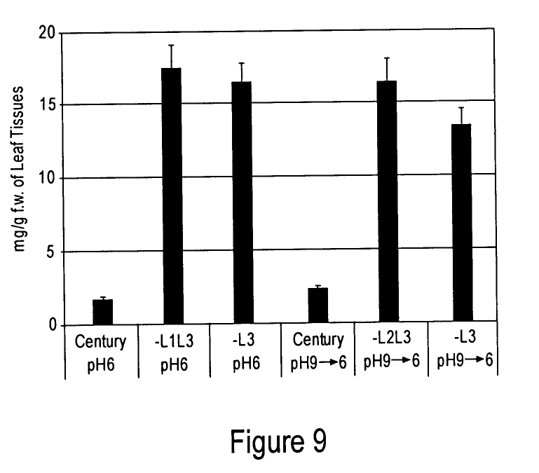

To further verify the effect of pH conditions on LOX isozyme activity, LOX1 and LOX2 as well as wild type soybean and LOX3 null lines were tested. For LOX2, 2.0 mg of 18:3 was homogenized with 10 mg of soy flour and 20 mg of leaf tissues in 2.5 mL of 100 mM pH 6.0 phosphate buffer for 2.0 minute. For LOX1, 18:3 and soy flour were homogenized in 2.0 mL of 50 mM K₂HPO₄ (pH8.7) for 1.0 minute, then the pH was lowered to about pH6.4 by the addition of 0.5 mL of 840 mM KH₂PO₄. The leaf tissues were added and homogenized for 2 minutes. The soy flour from the wild type Century as well as LOX3 null line was homogenized simultaneously with leaf tissues like LOX2 or sequentially like LOX1. As seen in FIG. 9, LOX2 (-L1 L3) at pH6 produced the highest level of C₆ aldehyde followed by LOX1 at pH9 and LOX3 null (contains both LOX1 and LOX2) at pH6, although there were no significant difference among them.

These results clearly showed the benefit of excluding LOX3 isozyme from the production mixture for optimal C₆ aldehyde production.

What is claimed is:

1. A method for enhancing the production of green note compounds comprising homogenizing green leaves of hydroperoxide producing plants in the presence of fatty acid and soy linoleate oxygen oxidoreductase (LOX), wherein the soy lipoxygenase is LOX1, LOX2 or a combination of LOX1 and LOX2.

2. The method of claim 1 wherein the green leaves are obtained from a hydroperoxide lyase (HL)-overproducing plant.

3. The method of claim 2 wherein the plants are genetically engineered HL-overproducing tobacco plants.

4. The method of claim 1 wherein the plant is a watermelon plant.

5. The method of claim 1 wherein the green leaves are obtained from a combination of different plants.

6. The method of claim 1 wherein the soy lipoxygenase is present in a soy bean seed flour or soy bean meal.

7. The method of claim 1 wherein the homogenizing is carried out in the presence of a buffer.

8. The method of claim 1 wherein the lipoxygenase is LOX1.

9. The method of claim 1 wherein the lipoxygenase is LOX2.

10. The method of claim 1 wherein the lipoxygenase is a mixture of LOX1 and LOX2.

11. The method of claim 1 wherein the green note compounds comprise C₆ aldehydes and alcohols.

12. The method of claim 1 wherein the fatty acid is linolenic acid, linoleic acid or a combination thereof.

13. The method of claim 1 wherein the fatty acid is a hydrolyzed oil rich in linolenic acid.

14. The method of claim 13 wherein the hydrolyzed oil is selected from linseed oil, sesame oil and soybean oil.

15. A method of enhancing the production of green note compounds comprising homogenizing green leaves of hydroperoxide producing plants in the presence of fatty acid and soy LOX 1, wherein a first step of homogenization of soyLOX1 and fatty acid is carried out in a buffer having a pH of about 9, followed by addition of plant tissues and buffer to lower the pH to about 6, and a second homogenization step.

16. A method of enhancing the production of green note compounds comprising homogenizing green leaves of hydroperoxide producing plants in the presence of fatty acid and soy LOX2, wherein the homogenizing is carried out in a buffer having a pH of about 6.

17. A method of enhancing the production of green note compounds comprising homogenizing green leaves of hydroperoxide producing plants in the presence of fatty acid and soy LOX1 and LOX2, wherein a first step of homogenization of soy LOX1, LOX2 and fatty acid is carried out in a buffer having a pH of about 9, followed by addition of plant tissues and buffer to lower the pH to about 6, and a second homogenization step.

18. The method of claim 17 comprising, wherein the homogenization is carried out in a buffer having a pH of about 6.

19. A soy bean seed flour or soy bean meal prepared from soy bean seeds that lack LOX3.

20. The soy bean seed flour or soy bean meal of claim 18 wherein the flour or meal comprises a single LOX isotype.

21. The soy bean seed flour or soy bean meal of claim 20 wherein the single isotype is LOX1.

22. The soy bean seed flour or soy bean meal of claim 20 wherein the single isotype is LOX2.

23. The soy bean seed flour or soy bean meal of claim 19 wherein the flour or meal comprises LOX1 and LOX2.
